(19) United States
(12) Patent Application Publication
(10) Pub. No.: US 2025/0046406 A1
(43) Pub. Date: Feb. 6, 2025

(54) CLAIM-LEVEL ERRONEOUS ELECTRONIC MEDICAL CLAIM RECORD DETECTION METHOD AND SYSTEM

(71) Applicant: M42 LTD, Abu Dhabi (AE)

(72) Inventors: Praveenkumar Kanithi, Masdar City (AE); Ronnie Rajan, Masdar City (AE); Shadab Khan, Masdar City (AE)

(73) Assignee: M42 LTD, Abu Dhabi (AE)

(21) Appl. No.: 18/365,548

(22) Filed: Aug. 4, 2023

(51) Int. Cl. G16H 10/60; G06F 40/40; G16H 40/20; G16H 50/20

(52) U.S. Cl. CPC G16H 10/60; G06F 40/40; G16H 40/20; G16H 50/20; G16H 20/10

(57) ABSTRACT

Methods, systems, and techniques for claim-level erroneous electronic medical claim record detection. An electronic medical claim record, which encodes a diagnosis for a patient by a medical services provider, is obtained. The diagnosis is input into a classifier trained using training diagnoses and training medical services provided in response to the training diagnoses. A predicted medical service provided by the medical services provider to the patient in response to the diagnosis is obtained as an output from the classifier.

### TECHNICAL FIELD

The present disclosure is directed at an erroneous electronic medical claim record detection method and system.

### BACKGROUND

When a medical services provider, such as a doctor, provides a medical service to a patient, the doctor will often store details of the medical service in an electronic medical claim record. These records are collected by certain data aggregators, such as insurance companies, which have an interest in ensuring that the records do not erroneously represent fraudulent activity. Practically, these data aggregators store and have access to electronic medical claim records representing a wide variety of medical services provided by a variety of different service providers at many different medical facilities. It would be beneficial to be able to process that data in a manner that practically allows erroneous electronic medical claim records to be detected.

### SUMMARY

According to a first aspect, there is provided a method comprising: obtaining electronic medical claim records, wherein the records encode medical services providers, medical activities performed by the medical services providers, and specialties of the medical services providers; identifying, from the electronic medical claim records, a cohort of the medical services providers by medical specialty; generating activity feature vectors respectively corresponding to the medical activities performed by the medical services providers of the cohort; determining a mixture model comprising components fit to the activity feature vectors; generating a provider feature vector for each of at least one of the medical services providers, wherein generating the provider feature vector comprises mapping activities performed by each of the at least one of the medical services providers in accordance with the mixture model; and processing each of the provider feature vectors using an anomaly detection method to identify the at least one of the medical services providers that have submitted abnormal electronic records.

The mixture model may comprise an unsupervised clustering method.

The unsupervised clustering method may comprise a Gaussian mixture model having sixteen components.

The anomaly detection method may comprise an unsupervised machine learning method.

The anomaly detection method may be selected from the group consisting of an isolation forest with a different number of trees, a one-class support vector machine, copula-based outlier detection, and scalable unsupervised outlier detection.

Generating the activity feature vectors may comprise converting a natural language representation of the medical activities into a vector embedding by applying a contextual word embedding model.

The contextual word embedding model may be a Bio-Clinical BERT model, and each of the medical activities may be converted into a vector embedding of length 768.

Generating the activity feature vectors may further comprise, before converting the natural language representation of the medical activities into the vector embedding, converting a numeric representation of the medical activities into the natural language representation.

Each the activities after the mapping may result in a membership vector, and generating the provider feature vector may further comprise aggregating the membership vectors corresponding to the activities by applying a Bag of Words model.

Generating the provider feature vector may further comprise combining, with the membership vectors, amounts claimed for performing the medical activities and comorbidity scores of patients to whom the medical activities were performed.

According to another aspect, there is provided a method comprising: obtaining an electronic medical claim record, wherein the record encodes a diagnosis for a patient by a medical services provider; inputting the diagnosis into a classifier trained using training diagnoses and training medical services provided in response to the training diagnoses; and obtaining, as output from the classifier, a predicted medical service provided by the medical services provider to the patient in response to the diagnosis.

The method may further comprise: comparing the predicted medical service to an actual medical service provided by the medical services provider to the patient in response to the diagnosis; and flagging the medical services provider as having an abnormal electronic record as a result of the actual and predicted medical services differing.

The classifier may output a predicted probability associated with the predicted medical service, and the comparing may comprise whether the predicted probability of the predicted medical service that corresponds to the actual medical service is above a predicted probability threshold.

The classifier may output a plurality of predicted medical services ranked by predicted probability of which the predicted medical service is a subset, and the comparing may comprise determining whether the actual medical service is within a top tier of the plurality of predicted medical services. The top tier may comprise a maximum threshold number of the ranked predicted medical services.

The diagnosis may be expressed in natural language, and the method may further comprise generating a diagnosis vector by converting the diagnosis from natural language into a vector embedding by applying a contextual word embedding model, and the classifier may output the predicted medical service based on the diagnosis vector.

The contextual word embedding model may be a Bio-Clinical BERT model, and the diagnosis may be converted into a vector embedding of length 768.

The method may further comprise generating a demographics vector representative of demographics of the patient, and the classifier may output the predicted medical service based on the diagnosis vector and on the demographics vector.

The demographics of the patient represented in the demographics vector may be selected from the group consisting of: patient gender, patient age, and patient risk score.

The demographics vector may be one-hot encoded.

The method may further comprise: processing the demographics vector using a multilayer perceptron network; concatenating the demographics vector after processing by the multilayer perceptron network with the vector embedding to result in a resulting vector; and inputting the resulting vector to a fully connected layer comprising part of the classifier to obtain the predicted medical service.

The multilayer perceptron network may be a 256×768, 2-layer network.

The training diagnoses and training medical services may be in respect of a specialization shared by the medical services provider.

The training medical services and predicted medical service may be limited to drug prescription.

The training medical services and predicted medical service may exclude drug prescription.

The classifier may comprise an XR-transformer.

The XR-transformer may comprise nodes each comprising a contextual word embedding model.

The contextual word embedding model may be a Bio-Clinical BERT model.

The output of the XR-transformer may comprise a one-hot encoded vector representing probabilities of predicted medical services for the diagnosis encoded in the electronic medical claim record.

According to another aspect, there is provided a method comprising: obtaining training diagnoses and training medical services provided in response to the training diagnoses; and using the training diagnoses and training medical services, training a classifier to output a predicted medical service in response to receiving as input a diagnosis for a patient by a medical services provider, wherein the diagnosis is encoded in an electronic medical claim record.

Each of the training diagnoses may be expressed in natural language, and the method may further comprise, for each of the training diagnoses, generating a diagnosis vector by converting the diagnosis from natural language into a vector embedding by applying a contextual word embedding model, and the classifier may output a training predicted medical service based on the diagnosis vector.

The contextual word embedding model may be a Bio-Clinical BERT model, and each of the training diagnoses may be converted into a vector embedding of length 768.

The method may further comprise generating a demographics vector representative of demographics of the patient, and the classifier may output the training predicted medical service based on the diagnosis vector and on the demographics vector.

The demographics of the patient represented in the demographics vector may be selected from the group consisting of: patient gender, patient age, and patient risk score.

The demographics vector may be one-hot encoded.

The method may further comprise: processing the demographics vector using a multilayer perceptron network; concatenating the demographics vector after processing by the multilayer perceptron network with the vector embedding to result in a resulting vector; and inputting the resulting vector to a fully connected layer comprising part of the classifier to obtain the training predicted medical service.

The multilayer perceptron network may be a 256×768, 2-layer network.

The training diagnoses may be in respect of a specialization shared by the medical services provider.

The training medical services may be limited to drug prescription.

The training medical services may exclude drug prescription.

The classifier may comprise an XR-transformer.

The XR-transformer may comprise nodes each comprising a contextual word embedding model.

The contextual word embedding model may be a Bio-Clinical BERT model.

An output of the XR-transformer may comprise a one-hot encoded vector representing probabilities of training predicted medical services for each of the training diagnoses.

According to another aspect, there is provided a system comprising: a database storing at least one electronic medical claim record; and a processor communicative with the database and configured to perform the foregoing method.

According to another aspect, there is provided a non-transitory computer readable medium having stored thereon computer program code that is executable by a processor and that, when executed by the processor, causes the processor to perform the foregoing method.

This summary does not necessarily describe the entire scope of all aspects. Other aspects, features and advantages will be apparent to those of ordinary skill in the art upon review of the following description of specific embodiments.

### BRIEF DESCRIPTION OF THE DRAWINGS

In the accompanying drawings, which illustrate one or more example embodiments:

FIG. 1 depicts a system for erroneous electronic medical claim record detection, according to an example embodiment.

FIG. 2 is a block diagram of a computer system that may be used in the system of FIG. 1.

FIG. 3 is a block diagram depicting how data is processed by when performing a method for erroneous electronic medical claim record detection, according to an example embodiment.

FIG. 4 is a flowchart depicting a method for erroneous electronic medical claim record detection, according to an example embodiment.

FIGS. 5A-5E and 6A-6E are graphs resulting from application of the method of FIG. 4.

FIG. 7 is a flowchart depicting a method for interfacing with the method of FIG. 4.

FIG. 8 is a flowchart depicting a method for erroneous electronic medical claim record detection, according to an example embodiment.

FIG. 9 is a block diagram depicting data flow in a method for erroneous electronic medical claim record detection, according to an example embodiment.

FIG. 10 depicts an architecture used in an XR-transformer of FIG. 9.

FIG. 11 depicts combination of a demographics vector and a diagnosis vector into a resulting vector for classification, according to an example embodiment.

### DETAILED DESCRIPTION

A medical services provider, such as a doctor or other clinician, typically records the nature of the services delivered to a patient in an electronic medical claim record (“EMCR”). The EMCR comprises information describing at least one claim, where each claim corresponds to a patient visit. For each claim, the EMCR typically comprises at least a unique claim identifier, the date of the visit, the provider's name, the patient's name, at least one diagnosis code respectively corresponding to at least one diagnosis (primary and/or secondary) made by the provider of the patient, and at least one activity code respectively corresponding to at least one medical activity (e.g., a treatment or test, such as prescriptions, lab tests, medication, imaging study, and/or equipment) ordered or performed by the provider for the patient. The EMCR may also comprise information such as the type of facility in which the at least one activity was provided (e.g., hospital, pharmacy, and/or diagnostics center), the city in which that facility is located, patient demographic information (e.g., age, weight, and/or gender), details pertaining to the provider's medical specialty, whether the patient is admitted to a hospital, and the amount the provider has claimed in respect of the visit. The information in the EMCR representing the at least one diagnosis and at least one activity may be encoded according to industry standards. For example, diagnoses may be encoded in accordance with the International Classification of Diseases (“ICD”) standard, and activities may be encoded using Current Procedural Terminology (“CPT”) codes.

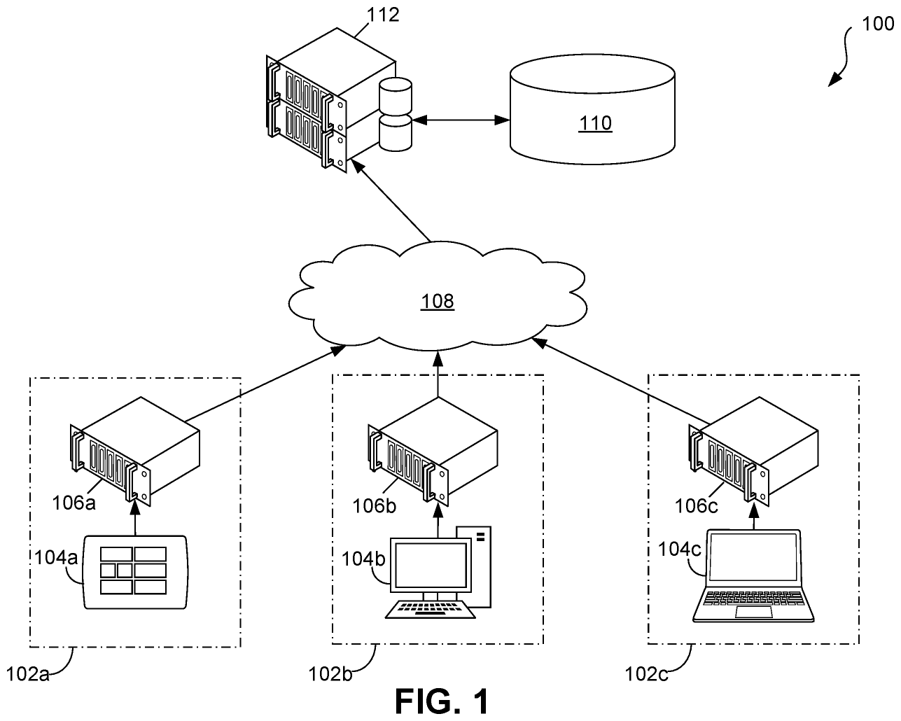

In order to be compensated, medical services providers submit claims to payers, such as insurance companies. These payers accordingly act as data aggregators that receive and process EMCRs from a wide variety of different facilities and providers and that compensate the providers based on the activities performed. This is depicted in FIG. 1, which is an example embodiment of a system 100 for processing EMCRs. The system 100 comprises first through third facilities 102a-c, such as hospitals, at which patient visits occur. The first through third facilities 102a-c comprise first through third provider client devices 104a-c that are communicative with first through third facility servers 106a-c, respectively. Following a patient visit, the provider responsible for that visit completes the corresponding EMCR using one of the provider client devices 104a-c, depending on which of the facilities 102a-c hosted the patient visit. A visit that occurs at the first facility 102a, for example, would result in the medical services provider completing the EMCR using the first provider client device 104a.

EMCRs input using the first through third provider client devices 104a-c are respectively uploaded to first through third facility servers 106a-c. The facility servers 106a-c process and locally store the EMCRs for their respective facility 102a-c and transmit, either periodically or in real-time, completed EMCRs to a data aggregator server 112 using a wide area network 108 such as the Internet. The data aggregator server 112 is controlled by a data aggregator such as an insurance company or a government. The data aggregator server 112 stores EMCRs it receives from the facilities 102a-c in a database 110, and processes and pays the medical services providers' claims based on the medical activities they performed as reflected in the EMCRs they submitted.

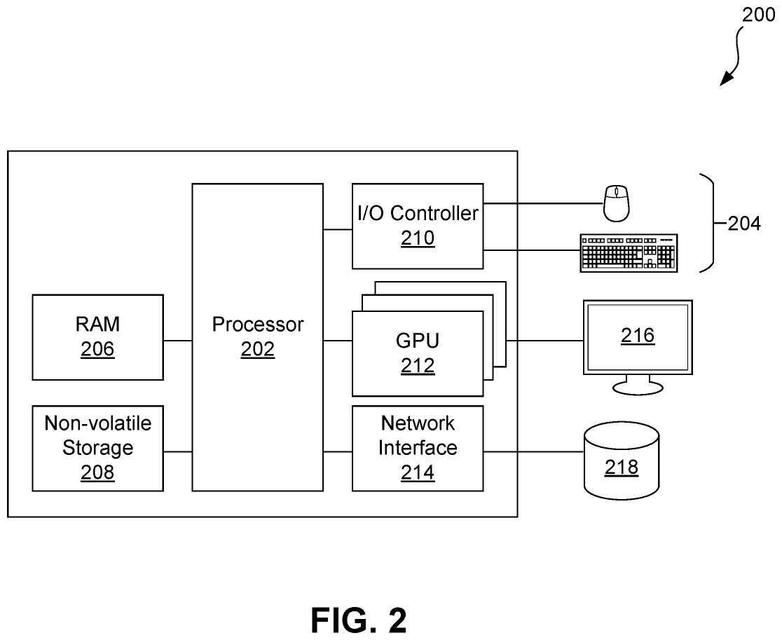

Each of the provider client devices 104a-c, facility servers 106a-c, and data aggregator server 112 may comprise a computer system 200 as depicted in FIG. 2. The computer system 200 comprises a processor 202 that controls the computer system's 200 overall operation. The processor 202 is communicatively coupled to and controls several subsystems. These subsystems comprise an input/output (“I/O”) controller 210, which is communicatively coupled to user input devices 204. The user input devices 204 may comprise, for example, any one or more of a keyboard, mouse, touch screen, and microphone. The subsystems further comprise random access memory (“RAM”) 206, which stores computer program code for execution at runtime by the processor 202; non-volatile storage 208, which stores the computer program code executed by the RAM 206 at runtime; graphical processing units (“GPU”) 212, which control a display 216 and which may be used to run one or more artificial neural networks in parallel; and a network interface 214, which facilitates network communications with a database 218. When the computer system 200 is used as the data aggregator server 112, the database 218 of FIG. 2 may be representative of the database 110 of FIG. 1; and when the computer system 200 is used as any of the data aggregator server 112 or the facility servers 106a-c, the network interface 214 may also be connected to the wide area network 108. The non-volatile storage 208 has stored on it computer program code that is loaded into the RAM 206 at runtime and that is executable by the processor 202. When the computer program code is executed by the processor 202, the processor 202 causes the computer system 200 to implement an appropriate method, such as a method for erroneous EMCR detection as described further below. Additionally or alternatively, multiple of the computer systems 200 may be networked together and collectively perform that method using distributed computing.

One technical problem faced by the data aggregator is how to identify aberrant data in the millions of EMCRs they typically process. Practically, aberrant data may correspond to fraud being perpetrated by certain medical services providers who have submitted claims in EMCRs. This type of erroneous EMCR may mean that “false upcoding” has occurred, which refers to fraudulently adding and/or changing ICD codes for a claim within an EMCR, typically to justify unnecessary prescriptions, procedures, and the like (this fraudulent adding and/or changing is “upcoding”). This adding or changing of ICD codes may be done, for example, to codes representing patient diagnoses and/or medical activities.

False upcoding is divided into “provider-level upcoding” and “claim-level upcoding”. Provider-level upcoding refers to medical services providers who repeatedly upcode diagnoses codes to increase the severity of the corresponding claims as a justification to charge for additional medical activities, or more expensive medical activities. Claim-level upcoding refers to unjustified activities being prescribed or performed by medical services provider for any given claim.

A data aggregator encounters significant technical problems when trying to identify false upcoding based on the ECMRs it receives from the facilities 102a-c. Namely, the data aggregator is presented with EMCRs representative of millions of claims, at least as many diagnoses and medical activities, without any labelling identifying which claims have been subject to upcoding. Identifying upcoding from this kind of dataset requires solving a technical problem; namely, how to leverage a computer to process high volumes of EMCRs without a priori information as to which of those EMCRs may be subject to upcoding. The embodiments herein are apply a particular solution that leverages unsupervised machine learning to solve this problem.

### Provider-Level Upcoding

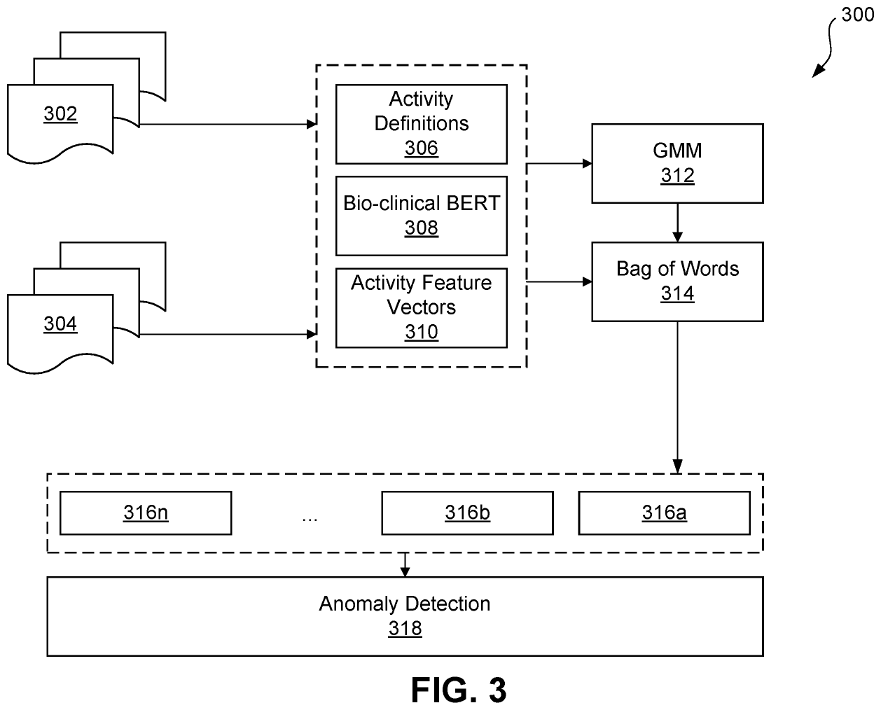

FIGS. 3, 4, 5A-5E, 6A-6E, and 7 are generally directed at provider-level upcoding.

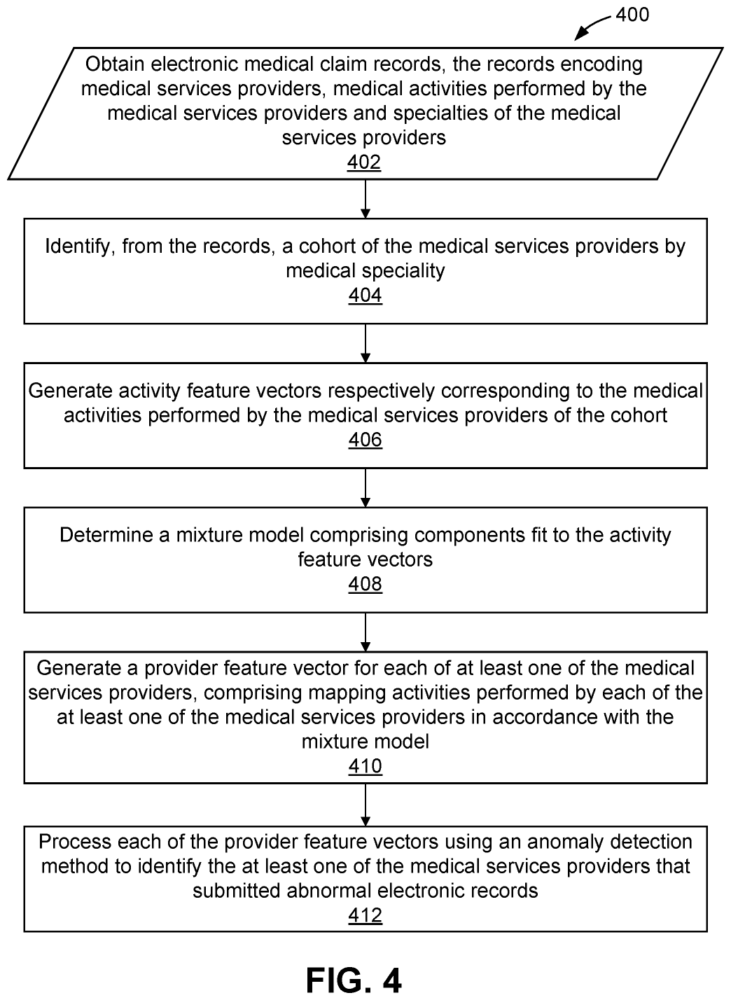

FIG. 3 is a block diagram 300 depicting how data is processed by when performing an example embodiment of a method 400 for erroneous electronic medical claim record detection, a flowchart of which is depicted in FIG. 4. The method 400 in this example embodiment is encoded as computer program code and performed, for example, by the data aggregator server 112. More particularly, the data aggregator server 112 may use a combination of its processor 202 and GPU(s) 212 to perform the method 400.

At block 402 of the method 400, the data aggregator server 112 obtains EMCRs, and in at least some instances corresponding metadata. As described above, the EMCRs encode claim information comprising medical services providers, medical activities performed by the medical services providers, and medical specialties of the medical services providers.

At block 404, the data aggregator server 112 identifies, from the EMCRs, a cohort of the medical services providers by medical specialty. A “cohort” of medical services providers refers to a grouping of medical services providers of the same, or similar, specialties, for example as identified in the EMCRs. In the present example embodiment, the method 400 is iteratively run with a view to generating feature vectors 316a-n for first through n^{th} medical services providers, respectively, with each run of the method 400 being directed at a particular one of the medical services providers belonging to a particular cohort. In FIG. 3, for example, the activities prescribed by the medical services provider who is the subject of a run of the method 400 (“current medical services provider”) are represented as provider-specific activities 304, while the activities prescribed by all of the medical services providers within that cohort are represented as cohort activities 302. Comparisons are made between a medical services provider and other medical services providers in the same cohort since medical services providers in different cohorts are expected to prescribe materially different medical activities, meaning little value would result from comparisons between providers of different cohorts.

A distribution of the cohort activities 302 is then modeled at blocks 406 and 408. At block 406, the data aggregator server 112 generates activity feature vectors 310 respectively corresponding to the medical activities performed by the medical services providers of the cohort. In the present example embodiment, each of the cohort activities 302 has a numerical representation corresponding, for example, to the activity's 302 CPT code, and that numeric representation is converted into a natural language representation. For example, when using CPT codes, activity 97761 is mapped to “Orthotic Management and Training and Prosthetic Training”. The natural language representations of the cohort activities 302 is represented in FIG. 3 as the activity definitions 306.

Once all the activities have been mapped to their natural language representations and the activity definitions 306 have consequently been created, each of the natural language representations is converted into a vector embedding by applying a contextual word embedding model. These vector embeddings, respectively corresponding to the activity definitions 306, act as the activity feature vectors 310. In FIG. 3, the contextual word embedding model is a Bio-clinical BERT model 308, and each of the medical activities is converted into a vector embedding of length 768. In at least some different embodiments, different language models and/or different vector embedding lengths may be used. The Bio-clinical BERT model 308 is trained on PubMed™ articles, as well as electronic health record data and discharge summaries from the MIMIC (Medical Information Mart for Intensive Care) dataset. A language model is used as opposed to a statistical model because language models capture the semantic relationships of the cohort activities 302; the more similar the cohort activities 302, the closer they are to each other in the embedding space.

At block 408, the data aggregator server 112 determines a mixture model comprising components fit to the activity feature vectors 310. In FIG. 3, a Gaussian mixture model (“GMM 312”) is determined. The GMM 312 is a probabilistic model that assumes all data points are generated from a mixture of a finite number of Gaussian distributions with unknown parameters. Following experiment, the GMM 312 of FIG. 3 was designed with 16 components. Fitting the GMM 312 to the activity feature vectors 310 results in respective membership vectors each having 16 components. The magnitude of each of the components represents the membership of the activity corresponding to the activity feature vector 310 to the respective component. Consequently, in the depicted embodiment if a membership vector does not have a dominant component, the activity corresponding to the activity feature vector 310 that is the basis for the membership vector is rarely prescribed by medical services providers in the cohort.

While the GMM 312 is used in FIG. 3, the GMM 312 is an example method of doing unsupervised clustering can in at least some other embodiments can be replaced by any suitable unsupervised clustering method (e.g., Fuzzy C-means clustering, spectral clustering, variational Gaussian mixture models, etc.). Additionally, while it was experimentally determined to use 16 components for the membership vector, the number of components may differ in at least some other embodiments and depend on modeling complexity. For example, a mixture model such as a variational Gaussian mixture model adaptively selects the best number of components to use.

At block 408, the data aggregator server 112 generates a provider feature vector for each of at least one of the medical services providers, who in the depicted embodiment is the current medical services provider and whose medical activities are represented as the provider-specific activities 304. In this example, generating the provider feature vector comprises mapping the provider-specific activities 304 in accordance with the GMM 312 to generate a respective number of membership vectors, as described above. The membership vectors in the depicted example embodiment are then aggregated by applying a Bag of Words model 314 and combined with metadata such as amounts claimed for performing the provider-specific activities 304 and comorbidity scores of patients to whom the medical provider-specific activities 304 were performed to result in the provider feature vector 316a for the current medical services provider. The comorbidity scores are determined based on the diagnosis information for each claim.

Once the data aggregator server 112 has determined the provider feature vector 316a, it processes the provider feature vector 316a using at least one anomaly detection method 318 to identify that the medical services provider has repeatedly submitted abnormal EMCRs. The at least one anomaly detection method 318 comprises in the depicted embodiment any suitable unsupervised machine learning method. Example unsupervised machine learning methods comprise an isolation forest with a different number of trees, a one-class support vector machine, copula-based outlier detection, and scalable unsupervised outlier detection. The output of the at least one anomaly detection method 318 is an upcoding score, which in the depicted embodiment is a final probability score representative of whether the current medical services provider made a fraudulent claim. When the at least one anomaly detection method 318 comprises more than one anomaly detection method 318, the upcoding score may represent an average of the probability scores respectively output by the more than one anomaly detection method 318. An upcoding score that satisfies an upcoding score threshold (e.g., 0.80, representing an 80% probability of an anomaly) is deemed to correspond to a fraudulent claim.

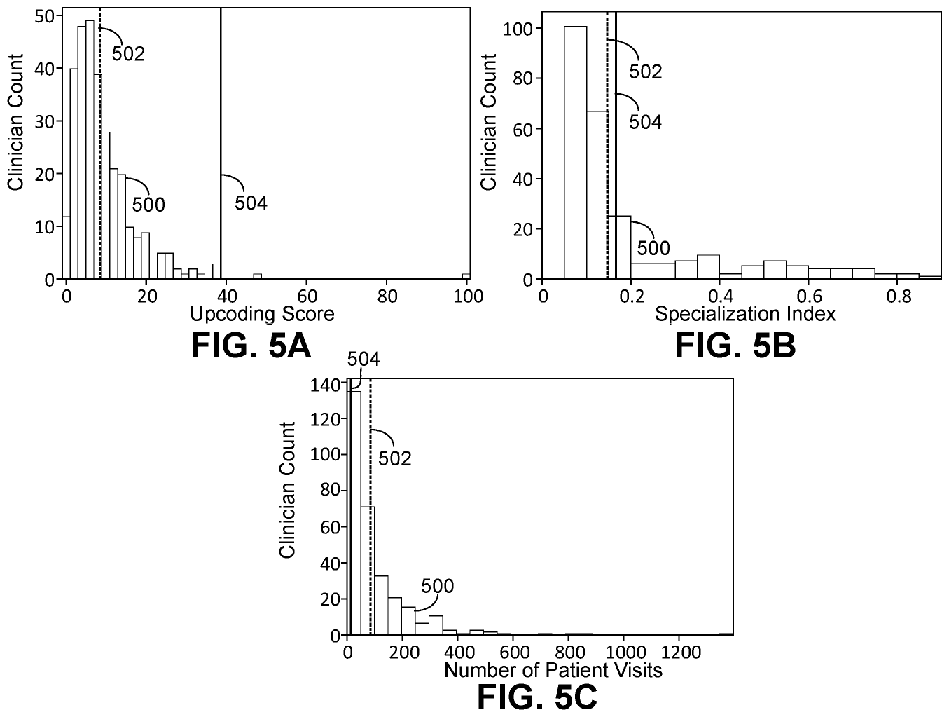

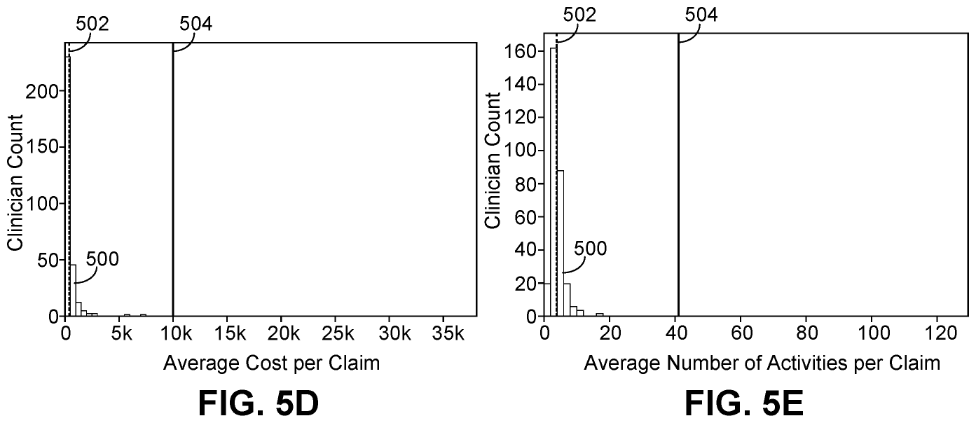

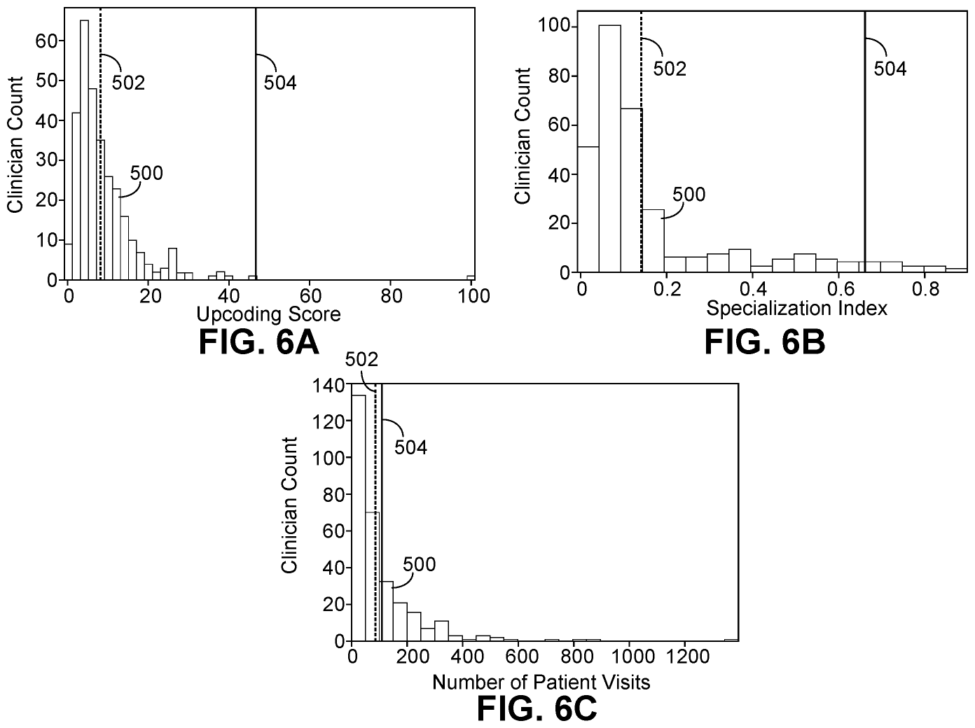

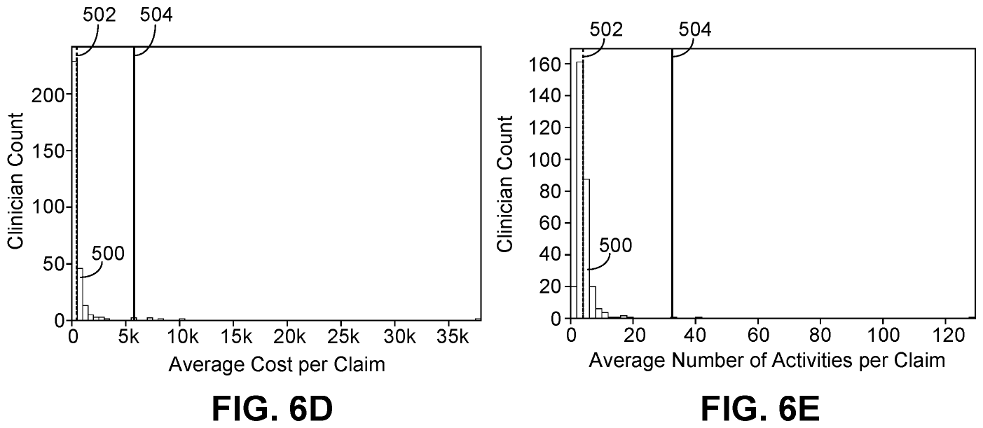

FIGS. 5A-5E and 6A-6E are graphs resulting from application of the method 400. More particularly, the graphs are all graphs of clinician (medical services provider) count vs. upcoding score (FIGS. 5A and 6A), specialization index (an indication of whether a clinician has specialized in the cohort of interest, determined by dividing the number of claims within the cohort of interest that were submitted by the clinician by the total number of claims made by that clinician) (FIGS. 5B and 6B), number of patient visits (FIGS. 5C and 6C), average cost per claim (FIGS. 5D and 6D), and average number of medical activities per claim (FIGS. 5E and 6E). Each of the graphs depict the plot 500 itself, a dotted line 502 representing the mean over all the clinicians, and a solid line 504 representing the current medical services provider. FIGS. 5A-5E are for one current medical services provider, and FIGS. 6A-6E are for another current medical services provider.

In FIG. 5A, the current medical services provider represented by the solid line 504 falls far from the average clinician as represented by the dashed line 502, implying a high likelihood of provider-level upcoding. Some other graphs reinforce this. For example, FIG. 5E shows the current medical services provider prescribed a large number of activities compared to the average clinician despite while having relatively few patient visits for the cohort of interest (FIG. 5C). And FIG. 5B shows that the current medical services provider has a specialization index close to the average clinician, implying there is no need for the current medical services provider to prescribe a large number of activities.

FIG. 6A also shows a current medical services provider whose upcoding score is relatively high. And FIG. 6E, similar to FIG. 5E, shows that the current medical services provider has also prescribed an unusually high number of activities. However, the current medical services provider has a high specialization index (FIG. 6B), which means the current medical services provider specializes in the cohort of interest and also gets more patient visits (FIG. 6C). Consequently, false coding is less likely than in FIGS. 5A-5E as the large number of activities and upcoding score may be justified by the high degree of specialization.

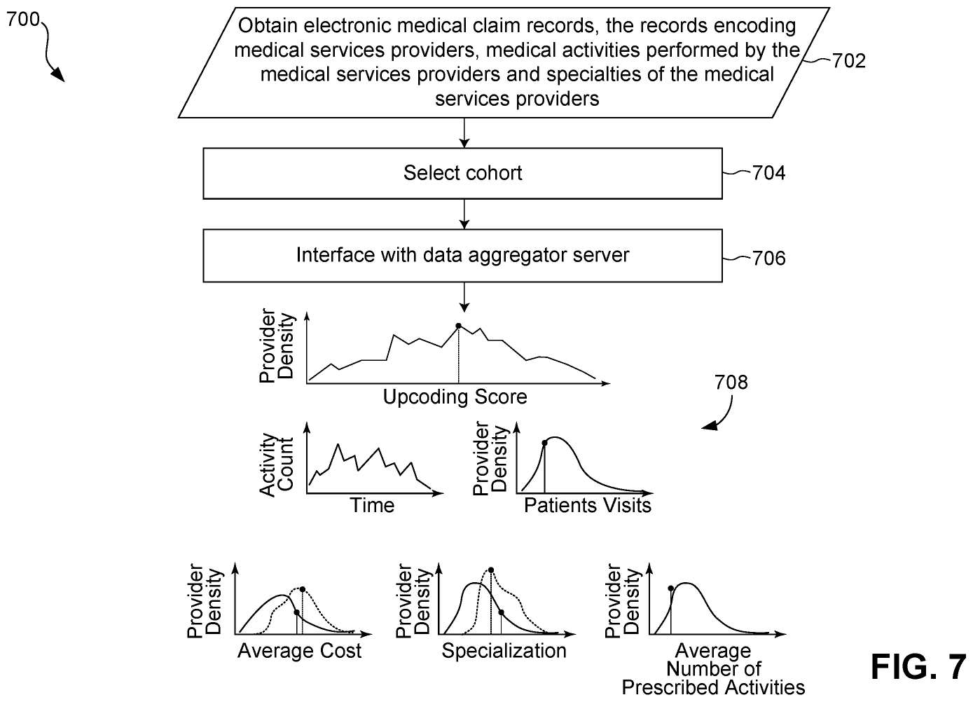

Referring now to FIG. 7, there is shown a method 700 for interfacing with the method 400 of FIG. 4, according to another example embodiment. A user interfacing with the data aggregator server 112 or a client of the data aggregator server 112 may perform the method 700. The user at block 702 obtains EMCRs, as referenced in block 402. At block 704, the user selects a cohort of interest. For example, the user may select a cohort using filters and a specific disease category. The user then interfaces with the data aggregator server 706, which has already performed the method 400 in respect of, for example, all of the medical services providers and has stored information related to which of those medical services providers is likely to have engaged in provider-level upcoding. The user may then cause the data aggregator server 706 to output information such as upcoding scores for any particular medical services provider in view of the selected cohort, and also obtain various plots 708 similar or analogous to the graphs of FIGS. 5A-5E and 6A-6E to assist in decision making.

In one example method of operation, the data aggregator server 112 and a user may perform the methods 400, 700 once every set number of days (e.g., once a week) to analyze new claims and to alert the user to medical services providers within selected cohort(s) who may have submitted erroneous EMCRs.

### Claim-Level Upcoding

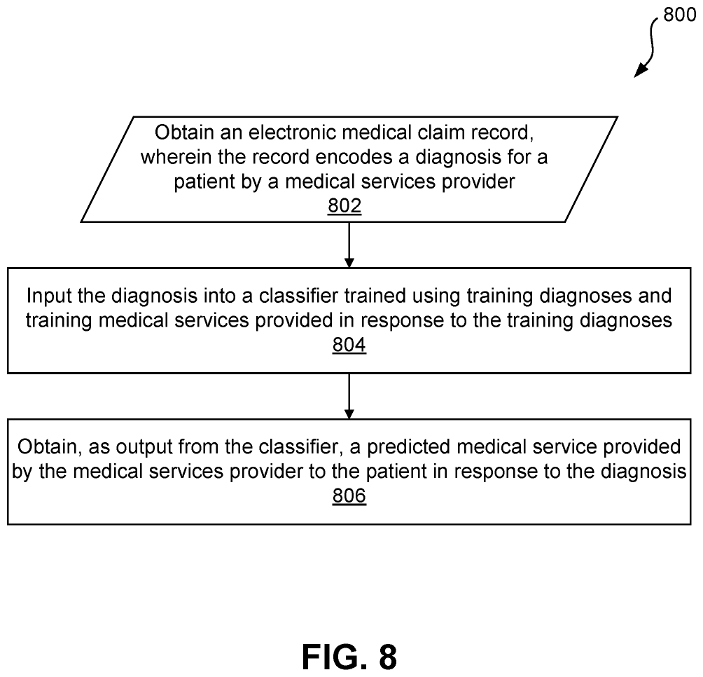

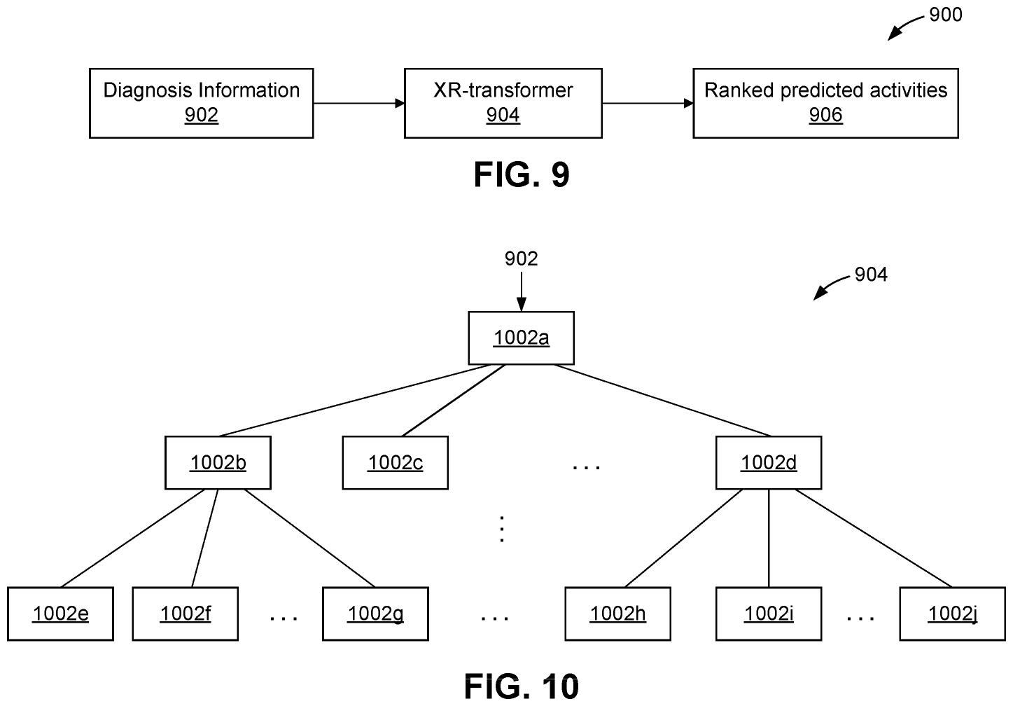

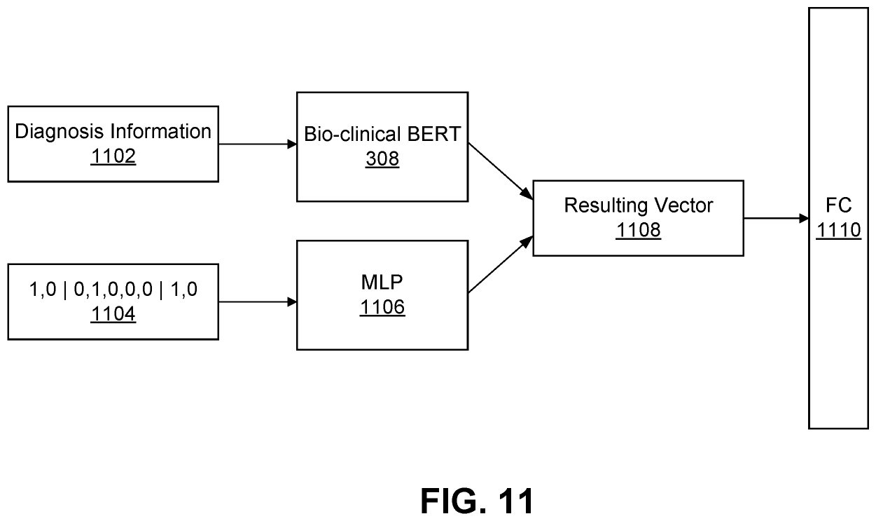

The system 100 of FIG. 1 may also be used to perform claim-level upcoding. Claim-level upcoding is described below in respect of FIGS. 8 and 9. FIG. 8 is a flowchart depicting a method 800 for erroneous electronic medical claim record detection, according to an example embodiment directed at claim-level upcoding. FIG. 9 depicts combination FIG. 9 depicts combination of a demographics vector and a diagnosis vector into a resulting vector for classification, which may be used in an example embodiment of the method 800 of FIG. 8. Claim-level upcoding is described below in conjunction with FIGS. 8 and 9.

As used herein, a “testing” data instance, such as a testing EMCR, refers to that data instance being used in conjunction with a classifier at inference, while a “training” data instance refers to a data instance used in conjunction with the classifier to train the classifier to perform that classification. A generic reference to a data instance may refer to using that instance in conjunction with testing and/or training, depending on the context.

The classifier used in conjunction with a first example embodiment (“XML embodiment”) of claim-level upcoding described herein comprises an XR-transformer, as referenced for example in Yu, H., Zhong, K., Zhang, J., Chang, W., and Dhillon, I. S., “PECOS: Prediction for Enormous and Correlated Output Spaces”, arXiv:2010.05878 [cs.LG], the entirety of which is hereby incorporated by reference herein. A benefit of using the XR-transformer is that, in the XML embodiment, the output dimension of the classifier corresponding to the number of medical activities can be on the order of tens of thousands, or millions. Consequently, an extreme machine learning (“XML”) framework is used, of which applying the XR-transformer is a part.

During training, historical claims data in the form of EMCRs are used as the training data. While there is a high chance that the training data is not perfect (e.g., it may contain mislabeled information), it is very likely that a significant majority of the claims represented in the training dataset are correct. Errors or noise in the training data may in some embodiments act as a regularization factor and prevent the classifier from being overfit to the training data.

As discussed above, the diagnoses comprising part of the EMCRs are encoded using, for example, ICD codes. Instead of one-hot encoding the diagnoses or using some statistical features as XR-transformer input, the diagnoses are encoded using the natural language definitions of ICD codes (e.g., ICD code S91.3 corresponds to “Open wound of foot”). Consequently, each input for the XR-transformer comprises at least one sentence, with each of the at least one sentence representing a natural language definition of the ICD diagnosis code in the EMCR. To differentiate between primary and secondary diagnoses, the primary diagnosis is presented as a first sentence as an input text paragraph to the XR-transformer with any secondary diagnoses following. The base model used for the XR-transformer comprises the Bio-clinical BERT 302, which as noted above has been trained on a large corpus of medical data and consequently generates more meaningful embeddings than a BERT trained on natural language generally. The output from the XR-transformer comprises a one-hot encoded vector of length equivalent to the number of activities in the training data.

Once trained, the classifier may be used to determine a predicted medical service provided by a medical services provider to a patient in response to the at least one diagnosis expressed in the EMCR for the patient's claim in accordance with the method 800 of FIG. 8. For example, the data aggregator server 112 may, at block 802, obtain the EMCR encoding at least one diagnosis for a patient by a medical services provider. In the present example, this at least one diagnosis comprises diagnoses encoded using ICD codes. The data aggregator server 112 generates an input text paragraph by converting the ICD codes into their natural language representations and by placing the primary diagnosis first in the input text paragraph. Once the input text paragraph is obtained, at block 804 the input text paragraph is input to the classifier comprising the XR-transformer trained using training diagnoses and training medical services provided in response to the training diagnoses according to a supervised learning model in accordance with the above description. At block 806, the data aggregator server 112 obtains as output from the classifier at least one predicted medical service provided by the medical services provider to the patient in response to the at least one diagnosis.

An example of this is depicted in FIGS. 9 and 10. FIG. 9 is a block diagram depicting data flow in the method 800 of FIG. 8, with an XR-transformer 904 used as a classifier, and FIG. 10 depicts an architecture used for the XR-transformer 904. More particularly, in FIG. 9, diagnosis information 902 represents the diagnoses encoded using, for example, ICD codes. These are input to an XR-transformer 904, the architecture for which is depicted in FIG. 10. FIG. 10 depicts an example subset of nodes 1002a-j, spanning portions of three example layers of the XR-transformer 904. As indicated in FIG. 10, the various layers of the XR-transformer 904 may comprise more than the depicted nodes 1002a-j, and the XR-transformer 904 may also comprise more than the three depicted layers.

The encoded diagnosis information 902 is input to a top layer comprising the first node 1002a. As shown in FIG. 10, the XR-transformer 904 is a hierarchical model in which each of the nodes 1002a-j comprises the Bio-clinical BERT 308 model with a number of classes equal to the number of children of that node, with those children comprising a subsequent layer of the XR-transformer 904. Each of the nodes 1002a-j of a final, output layer of the XR-transformer 904, which comprise the nodes labeled 1002e-j in FIG. 10, respectively map to activities in the training data. That is, each of the nodes 1002a-j of the output layer represent a possible predicted activity corresponding to the diagnosis information 902, with the value of the node 1002a-j representing the probability of that activity. These activities may be ranked based on those probabilities to result in ranked predicted activities 906 for FIG. 9.

In one example, the selected cohort has training data comprising 1,652,975 claims and test data comprising 20,000 claims with equal distribution of classes. The total number of medical services is 165,820. As part of the XML framework, labels are clustered at multiple levels to reduce the number of classes per stage. [64, 1,024, 16,384, 165,820] labels were used at each level and a multi-label classifier was trained for each stage using a single transformer model.

The one-hot encoded vector output by the classifier comprising the XR-transformer 904 represents predicted medical services for the diagnoses represented in the input text paragraph. As described above each of the predicted medical services has an associated probability. A hyperparameter k is selected (k may equal, for example, 15), and the top k predicted medical services are determined as relevant for the given input diagnoses. In order to flag a claim for potential upcoding, in one example embodiment if any one of the actual medical services provided by the medical services provider lies outside the top-k predicted medical services, those actual medical service(s) are flagged as corresponding to a potentially fraudulent claim and consequently erroneous EMCR. In this way, the classifier outputs a plurality of predicted medical services ranked by likelihood, and the data aggregator server 112 determines whether the actual medical service is within a top tier of the plurality of predicted medical services (e.g., within the top k predicted medical services), in which the top tier comprises a maximum threshold number (e.g., k) of the ranked predicted medical services as the total number of ranked predicted medical services may equal or exceed k.

Alternatively, if the actual medical service has less than predicted probability threshold in the output vector, the data aggregator server 112 may flag that actual medical service as being fraudulent regardless of whether it is within the top-k results (e.g., if k=15 and the actual medical service corresponds to k=10 with a predicted probability of 20% and the predicted probability threshold is 50%, the actual medical service may still be flagged as fraudulent).

To evaluate performance of the method 800, precision (what proportion of erroneous claims were in fact erroneous) and recall (what proportion of erroneously identified claims were in fact erroneous) were determined in accordance as follows:

$$Precision @ k = \frac{1}{k} {\sum }_{l = 1}^{k} {y}_{rank} \qquad ( l )$$

$$Recall @ k = \frac{1}{{\sum }_{i = 1}^{L} {y}_{i}} {\sum }_{l = 1}^{k} {y}_{rank} \qquad ( l )$$

where y∈{0, 1}^{L} is the ground truth label and rank (l) is the index of the l^{th} highest predicted label.

Table 1, below, shows precision and recall for different values of k (higher scores are better). The AUROC score was 87%.

TABLE 1
Example Method 800 Performance

| k | @1 | @2 | @3 | @4 | @5 |
|---|---|---|---|---|---|
| Precision | 23.25% | 20.00% | 18.14% | 16.83% | 15.82% |
| Recall | 15.11% | 23.01% | 28.41% | 32.57% | 35.98% |
| k | @6 | @7 | @8 | @9 | @10 |
| Precision | 14.84% | 14.06% | 13.32% | 12.75% | 12.21% |
| Recall | 38.78% | 41.35% | 43.49% | 45.53% | 47.41% |

In another example embodiment of claim-level upcoding (“reduced classes embodiment”), addition data pre-processing is done to reduce the total number of classes (corresponding to possible medical activities) to simplify the technical problem of applying the classifier to process EMCRs. Unlike in the XML framework described above in which the output space is extremely large and consequently an XR-transformer is applied, in the reduced classes embodiment separate models are built for medical activities that are 1) drug prescriptions and 2) all other procedures, with respect to each cohort (e.g., internal medicine, cardiology, dentistry, endocrinology); in the presently described embodiment, cohort is equated to specialization (i.e., the input data is filtered by specialization to arrive at the selected cohort), although in at least some other embodiments the cohort may be determined using one or more additional or alternative filters applied to the EMCRs. This reduces total number of classes per model, and also decouples model performance in respect of drug prescription from other medical activities across different specializations/cohorts. As evidenced below, this decoupling is done because non-drug medical activities tend to have better performance than drug prescription.

Sorting output classes by frequency, whether for drug prescription or other medical activities, results in a long-tail distribution. Consequently, relatively few classes account for the majority of claims in the EMCRs. Practically, low frequency medical activities are often highly specialized and generally are not representative of significant fraud or other errors. A threshold is accordingly set based on the most common medical activities, whether those activities are drug prescription or other medical activities. Doing this helps solve the technical machine learning problem of how to process long-tail distributions, which is difficult due to severe data imbalance.

FIG. 9 depicts the data pre-processing referenced above. Diagnosis information 1102 represents primary and secondary diagnosis information, as described above in respect of the XML embodiment. A demographics vector 1104 is representative of demographics of the patient for any particular claim. Example demographics information encoded in the demographics vector 1104 are patient gender, patient age, and patient risk score, where the patient risk score may be the Charlson risk score. The demographics vector 1104 in FIG. 9 is one-hot encoded, although it may be differently encoded in different embodiments. When one-hot encoded, certain demographics information such as age and risk score may be divided into multiple ranges. Regarding the risk score in particular, the risk score is divided into a pre-defined number of bins and converted into a one-hot vector based on the bin into which it falls. For example, if the risk score is normalized to [0,1], the bins may be represented by the intervals [0, 0.2), [0.2, 0.4), [0.4, 0.6), [0.6, 0.8), [0.8, 1.0), and a risk score of 0.3 is accordingly converted into the one-hot vector of [0, 1, 0, 0, 0] where “1” represents the bin into which 0.3 falls. The pre-processing depicted in FIG. 9 is performed on a per selected cohort basis, with each cohort having a relatively reduced number of classes relative to the XML embodiment. As patient demographic information does not change with clinician cohort, the one-hot encoded demographics vector 1104 is does not change with the selected cohort.

The diagnosis information 1102 is input to the Bio-clinical BERT 308 to result in a diagnosis vector (not shown). The Bio-clinical BERT 308 converts the diagnosis information from natural language into a vector embedding of length 768 by applying a contextual word embedding model. In at least some other embodiments, the vector may have a different number of embeddings, and and/or a different contextual word embedding model may be used.

FIG. 9 also shows that the demographics vector 1104 is processed using a multilayer perceptron network (“MLP 1106”). The example MLP 1106 of FIG. 9 is a 256×768, 2-layer network; however, in at least some other embodiments a differently dimensioned multilayer perceptron network may be used depending, for example, on data, cohort, and the like. The processed demographics vector 1104 as output by the MLP 768 and the diagnosis vector output by the Bio-clinical BERT 308 are concatenated into a resulting vector 1108. The resulting vector 1108 is input to a fully connected layer 1110 to obtain the predicted medical service or a list of at least k predicted services as described above. The fully connected layer 1110 has a number of output neurons equal to the number of medical activities to be predicted.

The classifier in the reduced classes embodiment of FIG. 9 comprises the Bio-clinical BERT 302, MLP 1106, and fully connected layer 1110; consequently, training is done on them in a manner analogous to any neural network by back propagating loss through the Bio-clinical BERT 302 and MLP 1106 using the chain rule.

Experiments were performed in which patient data was split into train, validation, and test with respect to each medical specialization/cohort. Unlike a random stratified split performed with the XML framework described above, in the reduced classes embodiment a temporal split was performed for each cohort. In other words, the data was ordered according to the dates on which medical services were provided and the most recent ˜30,000 claims were selected for each test, the next most recent ˜30,000 claims were selected for validation, and the remaining claims were used for training. This type of data split helps replicate real-world working conditions.

Results corresponding to each specialization are below using the precision and recall metrics described above in respect of the XML embodiment. For data pre-processing, a threshold of 10 classes was set for non-drug prescription medical activities and 50 for drug prescription activities to remove long tails. As the resulting data varies with respect to each cohort, the number of output classes for each model also varies and is given below.

Below, Table 2 provides results endocrinology (˜360k claims for training), Table 3 for cardiology (˜640k claims for training), Table 4 for dental (˜1M claims for training), and Table 5 for internal medicine (˜2M claims for training). “Drugs” in Tables 2-5 refers to drug prescription, while “Procedures” refers to all other medical activities.

TABLE 2
Endocrinology Test Results

| | | | |
|---|---|---|---|
| k | 1 | 5 | 10 |

Procedures (371 classes)

| | | | |
|---|---|---|---|
| Precision | 0.973 | 0.628 | 0.441 |
| Recall | 0.173 | 0.535 | 0.679 |

Drugs (528 classes)

| | | | |
|---|---|---|---|
| Precision | 0.962 | 0.589 | 0.305 |
| Recall | 0.147 | 0.501 | 0.618 |

TABLE 3
Cardiology Test Results

| | | | |
|---|---|---|---|
| k | 1 | 5 | 10 |

Procedures (408 classes)

| | | | |
|---|---|---|---|
| Precision | 0.977 | 0.625 | 0.432 |
| Recall | 0.193 | 0.604 | 0.751 |

Drugs (1104 classes)

| | | | |
|---|---|---|---|
| Precision | 0.968 | 0.601 | 0.326 |
| Recall | 0.161 | 0.522 | 0.666 |

TABLE 4
Dental Test Results

| | | | |
|---|---|---|---|
| k | 1 | 5 | 10 |

Procedures (612 classes)

| | | | |
|---|---|---|---|
| Precision | 0.983 | 0.598 | 0.406 |
| Recall | 0.201 | 0.651 | 0.783 |

Drugs (811 classes)

| | | | |
|---|---|---|---|
| Precision | 0.971 | 0.512 | 0.234 |
| Recall | 0.141 | 0.496 | 0.577 |

TABLE 5
Internal Medicine Test Results

| | | | |
|---|---|---|---|
| k | 1 | 5 | 10 |

Procedures (616 classes)

| | | | |
|---|---|---|---|
| Precision | 0.986 | 0.695 | 0.508 |
| Recall | 0.162 | 0.532 | 0.703 |

Drugs (2,874 classes)

| | | | |
|---|---|---|---|
| Precision | 0.986 | 0.597 | 0.337 |
| Recall | 0.201 | 0.605 | 0.676 |

From Tables 2-4, a user can set the value of k as desired. Precision at k=1 for all the results in Tables 2-4 is around 0.98 in all cases, which shows 98% of the time the reduced classes embodiment's most confident predicted activity is the correct prescribed activity.

Below are examples of textual input and output generated from the data aggregator server 112 of the reduced classes embodiment, which show example with diagnoses information from an example EMCR, ground truth, and the top 15 predicted medical activities (i.e., k=15).

In a first example:
(i) Diagnoses information: Enlarged prostate without lower urinary tract symptoms, Type 2 diabetes mellitus without complications, Hyperlipidemia, unspecified Allergic rhinitis, unspecified Chronic obstructive pulmonary disease, unspecified Essential (primary) hypertension
(ii) Ground truth:
   LIPID PANEL
   Transferase; aspartate amino (AST) (SGOT)
   Urinalysis, by dip stick or tablet reagent for bilirubin, glucose, hemoglobin, ketones, leukocytes, nitrite, pH, protein, specific gravity, urobilinogen, any number of these constituents; automated, with microscopy
   Triglycerides
(iii) Top 15 predicted labels:
   LIPID PANEL
   Urinalysis, by dip stick or tablet reagent for bilirubin, glucose, hemoglobin, ketones, leukocytes, nitrite, pH, protein, specific gravity, urobilinogen, any number of these constituents; automated, with microscopy
   Transferase; aspartate amino (AST) (SGOT)
   Thyroid stimulating hormone (TSH)
   25 HYDROXY INCLUDES FRACTIONS IF PERFORMED
   BLOOD COUNT COMPLETE AUTO&AUTO DIFRNTL WBC COUNT
   TRANSFERASE ALANINE AMINO
   Hemoglobin; glycosylated (A1C)
   THYROXINE FREE
   Uric acid; blood
   HPYLORI STOOL IA
   Folic acid; serum
   UREA NITROGEN QUANTITATIVE
   Triglycerides
   URINE ALBUMIN QUANTITATIVE

In a second example:
(i) Diagnoses information: Essential (primary) hypertension, Vitamin B12 deficiency anemia, unspecified Mixed hyperlipidemia
(ii) Ground Truth:
   OFFICE OUTPATIENT VISIT 15 MINUTES
   LIPID PANEL
   BLOOD COUNT COMPLETE AUTO&AUTO DIFRNTL WBC COUNT
   POTASSIUM SERUM PLASMA/WHOLE BLOOD
   BILIRUBIN TOTAL
(iii) Top 15 predicted labels:
   POTASSIUM SERUM PLASMA/WHOLE BLOOD
   LIPID PANEL
   BLOOD COUNT COMPLETE AUTO&AUTO DIFRNTL WBC COUNT
   OFFICE OUTPATIENT VISIT 15 MINUTES
   ECHO TTHRC R-T 2D W/WOM-MODE COMPL SPEC&COLR DOP
   Thyroid stimulating hormone (TSH)
   Hemoglobin; glycosylated (A1C)
   Transferase; aspartate amino (AST) (SGOT)
   CARDIOVASCULAR STRESS TEST
   Troponin, quantitative
   OFFICE OUTPATIENT NEW 30 MINUTES
   1 free F/U allowed within 7 days from Discharge/initial OP Consultation
   Consultant Fee-OPD
   URINE ALBUMIN QUANTITATIVE
   UREA NITROGEN QUANTITATIVE

The embodiments have been described above with reference to flow, sequence, and block diagrams of methods, apparatuses, systems, and computer program products. In this regard, the depicted flow, sequence, and block diagrams illustrate the architecture, functionality, and operation of implementations of various embodiments. For instance, each block of the flow and block diagrams and operation in the sequence diagrams may represent a module, segment, or portion of code, which comprises one or more executable instructions for implementing the specified action(s). In some alternative embodiments, the action(s) noted in that block or operation may occur out of the order noted in those figures. For example, two blocks or operations shown in succession may, in some embodiments, be executed substantially concurrently, or the blocks or operations may sometimes be executed in the reverse order, depending upon the functionality involved. Some specific examples of the foregoing have been noted above but those noted examples are not necessarily the only examples. Each block of the flow and block diagrams and operation of the sequence diagrams, and combinations of those blocks and operations, may be implemented by special purpose hardware-based systems that perform the specified functions or acts, or combinations of special purpose hardware and computer instructions.

The terminology used herein is for the purpose of describing particular embodiments only and is not intended to be limiting. Accordingly, as used herein, the singular forms “a”, “an”, and “the” are intended to include the plural forms as well, unless the context clearly indicates otherwise (e.g., a reference in the claims to “a challenge” or “the challenge” does not exclude embodiments in which multiple challenges are used). It will be further understood that the terms “comprises” and “comprising”, when used in this specification, specify the presence of one or more stated features, integers, steps, operations, elements, and components, but do not preclude the presence or addition of one or more other features, integers, steps, operations, elements, components, and groups. Directional terms such as “top”, “bottom”, “upwards”, “downwards”, “vertically”, and “laterally” are used in the following description for the purpose of providing relative reference only, and are not intended to suggest any limitations on how any article is to be positioned during use, or to be mounted in an assembly or relative to an environment. Additionally, the term “connect” and variants of it such as “connected”, “connects”, and “connecting” as used in this description are intended to include indirect and direct connections unless otherwise indicated. For example, if a first device is connected to a second device, that coupling may be through a direct connection or through an indirect connection via other devices and connections. Similarly, if the first device is communicatively connected to the second device, communication may be through a direct connection or through an indirect connection via other devices and connections. The term “and/or” as used herein in conjunction with a list means any one or more items from that list. For example, “A, B, and/or C” means “any one or more of A, B, and C”.

It is contemplated that any part of any aspect or embodiment discussed in this specification can be implemented or combined with any part of any other aspect or embodiment discussed in this specification, so long as those parts are not mutually exclusive.

The scope of the claims should not be limited by the embodiments set forth in the above examples, but should be given the broadest interpretation consistent with the description as a whole.

It should be recognized that features and aspects of the various examples provided above can be combined into further examples that also fall within the scope of the present disclosure. In addition, the figures are not to scale and may have size and shape exaggerated for illustrative purposes.

What is claimed is:

1. A method comprising:
(a) obtaining an electronic medical claim record, wherein the record encodes a diagnosis for a patient by a medical services provider;
(b) inputting the diagnosis into a classifier trained using training diagnoses and training medical services provided in response to the training diagnoses; and
(c) obtaining, as output from the classifier, a predicted medical service provided by the medical services provider to the patient in response to the diagnosis.

2. The method of claim 1, further comprising:
(a) comparing the predicted medical service to an actual medical service provided by the medical services provider to the patient in response to the diagnosis; and
(b) flagging the medical services provider as having an abnormal electronic record as a result of the actual and predicted medical services differing.

3. The method of claim 2, wherein the classifier outputs a predicted probability associated with the predicted medical service, and wherein the comparing comprises whether the predicted probability of the predicted medical service that corresponds to the actual medical service is above a predicted probability threshold.

4. The method of claim 2, wherein the classifier outputs a plurality of predicted medical services ranked by predicted probability of which the predicted medical service is a subset, and wherein the comparing comprises determining whether the actual medical service is within a top tier of the plurality of predicted medical services, wherein the top tier comprises a maximum threshold number of the ranked predicted medical services.

5. The method of claim 1, wherein the diagnosis is expressed in natural language, wherein the method further comprises generating a diagnosis vector by converting the diagnosis from natural language into a vector embedding by applying a contextual word embedding model, and wherein the classifier outputs the predicted medical service based on the diagnosis vector.

6. The method of claim 5, wherein the contextual word embedding model is a Bio-Clinical BERT model, and wherein the diagnosis is converted into a vector embedding of length 768.

7. The method of claim 5, further comprising generating a demographics vector representative of demographics of the patient, and wherein the classifier outputs the predicted medical service based on the diagnosis vector and on the demographics vector.

8. The method of claim 7, wherein the demographics of the patient represented in the demographics vector is selected from the group consisting of: patient gender, patient age, and patient risk score.

9. The method of claim 7, wherein the demographics vector is one-hot encoded.

10. The method of claim 7, further comprising:
(a) processing the demographics vector using a multilayer perceptron network;
(b) concatenating the demographics vector after processing by the multilayer perceptron network with the vector embedding to result in a resulting vector; and
(c) inputting the resulting vector to a fully connected layer comprising part of the classifier to obtain the predicted medical service.

11. The method of claim 10, wherein the multilayer perceptron network is a 256×768, 2-layer network.

12. The method of claim 1, wherein the training diagnoses and training medical services are in respect of a specialization shared by the medical services provider.

13. The method of claim 1, wherein the training medical services and predicted medical service are limited to drug prescription.

14. The method of claim 1, wherein the training medical services and predicted medical service exclude drug prescription.

15. The method of claim 1, wherein the classifier comprises an XR-transformer.

16. The method of claim 15, wherein the XR-transformer comprises nodes each comprising a contextual word embedding model.

17. The method of claim 16, wherein the contextual word embedding model is a Bio-Clinical BERT model.

18. The method of claim 16, wherein an output of the XR-transformer comprises a one-hot encoded vector representing probabilities of predicted medical services for the diagnosis encoded in the electronic medical claim record.

19. A system comprising:
(a) a database storing at least one electronic medical claim record; and
(b) a processor communicative with the database and configured to perform a method comprising:
(i) obtaining the electronic medical claim record, wherein the record encodes a diagnosis for a patient by a medical services provider;
(ii) inputting the diagnosis into a classifier trained using training diagnoses and training medical services provided in response to the training diagnoses; and
(iii) obtaining, as output from the classifier, a predicted medical service provided by the medical services provider to the patient in response to the diagnosis.

20. A non-transitory computer readable medium having stored thereon computer program code that is executable by a processor and that, when executed by the processor, causes the processor to perform a method comprising:
(a) obtaining an electronic medical claim record, wherein the record encodes a diagnosis for a patient by a medical services provider;
(b) inputting the diagnosis into a classifier trained using training diagnoses and training medical services provided in response to the training diagnoses; and
(c) obtaining, as output from the classifier, a predicted medical service provided by the medical services provider to the patient in response to the diagnosis.
